(19) United States
(12) Patent Application Publication
(10) Pub. No.: US 2006/0199781 A1
(43) Pub. Date: Sep. 7, 2006

(54) Assays based on BTF3 activity

(75) Inventors: Joel H. Rothman, Santa Barbara, CA (US); Tim Bloss, Oxnard, CA (US); Eric Witze, Santa Barbara, CA (US)

Correspondence Address: PETERS VERNY JONES & SCHMITT, L.L.P., 425 SHERMAN AVENUE, SUITE 230, PALO ALTO, CA, 94306, US

(73) Assignee: The Regents of the University of California

(21) Appl. No.: 11/305,405

(22) Filed: Dec. 16, 2005

Related U.S. Application Data
(62) Division of application No. 10/153,344, filed on May 21, 2002, now abandoned.
(60) Provisional application No. 60/292,559, filed on May 21, 2001.

(51) Int. Cl. A61K 48/00; C12Q 1/68; G01N 33/567; A61K 39/395; G01N 33/53

(52) U.S. Cl. 514/44; 424/133.1; 435/6; 435/7.2

(57) ABSTRACT

This invention pertains to the discovery that BTF3 plays a critical, negative-regulatory role in programmed cell death (PCD) in C. elegans and other species. Overexpression of BTF3 leads to decreased programmed cell death, while inactivation of BTF3 leads to increased programmed cell death. Methods of modulating (upregulating or downregulating) programmed cell death by increasing or decreasing expression and/or activity of BTF3 are provided. These methods are useful in the treatment of various pathologies including, but not limited to cancer and neurodegenerative diseases.

### CROSS-REFERENCE TO RELATED APPLICATIONS

This application claims priority to and benefit of U.S. Ser. No. 60/292,559, filed on May 21, 2001, and is a divisional of U.S. Ser. No. 10/153,344, filed May 21, 2002, which applications are incorporated herein by reference in its entirety for all purposes.

### STATEMENT OF GOVERNMENTAL SUPPORT

This work was supported by grants from the National Institutes of Health, National Institute on Aging and Institute of Child Health and Human Disease (NICHD), and a March of Dimes Birth Defects Foundation grant. The Government of the United States of America may have certain rights in this invention.

### REFERENCE TO SEQUENCE LISTING

Applicants assert that the paper copy of the Sequence Listing is identical to the Sequence Listing in computer readable form found on the accompanying computer disk. Applicants incorporate the contents of the sequence listing by reference in its entirety.

### FIELD OF THE INVENTION

The present invention relates to the field of apoptosis. In particular, this invention pertains to the discovery that BTF3 and its homologues prevent “programmed” cell death (apoptosis).

### BACKGROUND OF THE INVENTION

Many diseases are associated with excessive apoptosis. For example, HIV leads to AIDS by promoting apoptotic death of CD4+ T cells. A range of neurodegenerative diseases (e.g., ALS, spinal muscular atrophy, and Alzheimer's and Parkinson's diseases) are the result of progressive death of neurons. Ischemia is associated with apoptotic death of cardiomyocytes and neurons during progression of myocardial infarction and stroke.

Inappropriate repression of programmed cell death can also result in a wide array of diseases. Productive viral infection often requires active repression of programmed cell death by a virally encoded product (e.g. E6 of human papilloma virus binding to and inactivating p53, BARF1 of Epstein-Barr virus upregulating the expression of the anti-apoptotic factor Bcl-2). Auto-reactive lymphocytes are normally eliminated by programmed cell death and autoimmune diseases such as lupus erythematosus can arise by a failure of such cells to die.

The failure of cells to undergo programmed cell death is implicated in tumorigenesis in a variety of human malignancies. Cells that have accumulated high levels of DNA damage are eliminated from the organism via programmed cell death without negatively affecting the surrounding tissue. Disruption of programmed cell death in a cell greatly increases the chance of that cell becoming tumorgenic, since the damage can cause mutations that lead to malignant transformation. In addition, programmed cell death appears to be a first line of defense against the proliferation of cells that might form a tumor: cells in which growth control is dysregulated in a way that could result in uncontrolled proliferation are generally able to recognize that aberrant state and commit suicide by programmed cell death. If programmed cell death is blocked in such cells, cancer could arise. The failure to undergo programmed cell death per se can even lead to excessive numbers of cells and cancer: e.g., as the result of inappropriate activation of the bcl-2 gene, a suppressor of programmed cell death, most follicular B cell lymphomas result in the accumulation of excessive number of cells that would normally undergo programmed cell death. Many tumor cell types also appear to require bcl-2 expression to avoid apoptosis and remain proliferative. Thus, the inability to regulate programmed cell death may be a key causative event in many, and perhaps all, cancers.

### SUMMARY OF THE INVENTION

This invention pertains to the discovery that Cenorhabditis elegans BTF3 (Ce-BTF3) plays a critical, negative-regulatory role in programmed cell death (PCD) in C. elegans and that analogs of BTF3 show similar roles in other organisms. Overexpression of Ce-BTF3 leads to decreased programmed cell death, while inactivation of Ce-BTF3 leads to increased programmed cell death. We have identified a putative CARD region on Ce-BTF3 that we believe is involved in the regulation of apoptosis through direct (or indirect) association with the caspase CED-3, the protein thought to be required for all programmed cell death in C. elegans.

Assays are provided to screen for agents that alter BTF3 experssion and/or activity and thereby modulate (increase or decrease) programmed cell death. In addition, methods are provided for increasing programmed cell death by decreasing BTF3 expression and/or activity. Methods are provided for decreasing programmed cell death by increasing BTF3 expression and/or activity.

In one embodiment, this invention provides a method of inhibiting programmed cell death of a cell. The method involves upregulating expression or activity of BTF3 or a BTF3 homologous in the cell. The upregulating can be by any convenient method (e.g. upregulating the expression of endogenous BTF3 by the use of a compound that upregulates BTF3 expression, by modification of a BTF3 promoter or enhancer, etc.). In certain instances the upregulating comprises transfecting a cell with a nucleic acid that encodes a BTF3 polypeptide. In certain embodiments, the upregulating comprises transfecting the cell with a BTF3 polypeptide.

In another embodiment, this invention provides a method of increasing programmed cell death of a cell. The method involves inhibiting expression or activity of BTF3 or a BTF3 homologue in the cell. The inhibition can be by any number of methods. In certain embodiments, the inhibiting comprises contacting a BTF3 nucleic acid (e.g. a BTF3 RNA) with an antisense oligonucleotide. In certain embodiments, the inhibiting comprises contacting a BTF3 nucleic acid with a ribozyme and/or a catalytic DNA that specifically cleaves said BTF3 nucleic acid or transfecting the cell with an inhibitory RNA (i.e. RNAi). In certain embodiments, the inhibiting comprises transfecting the cell comprising a BTF3 gene with a nucleic acid that inactivates the BTF3 gene by homologous recombination with the BTF3 gene, the BTF3 promoter, or intervening nucleic acids. In certain embodiments, the inhibiting comprises transfecting a cell comprising a BTF3 gene with a nucleic acid encoding an intrabody that specifically binds a BTF3 polypeptide and/or contacting a cell comprising an BTF3 gene with a small organic molecule that inhibits expression of the BTF3 gene. In certain embodiments, the cell is a hyperproliferative cell (e.g. a cancer cell). Such cancer cells include, but are not limited to cells of a cancer selected from the group consisting of a lung cancer, a bronchus cancer, a colorectal cancer, a prostate cancer, a breast cancer, a pancreas cancer, a stomach cancer, an ovarian cancer, a urinary bladder cancer, a brain or central nervous system cancer, a peripheral nervous system cancer, an esophageal cancer, a cervical cancer, a melanoma, a uterine or endometrial cancer, a cancer of the oral cavity or pharynx, a liver cancer, a kidney cancer, a biliary tract cancer, a small bowel or appendix cancer, a salivary gland cancer, a thyroid gland cancer, a adrenal gland cancer, an osteosarcoma, a chondrosarcoma, a liposarcoma, and a testes cancer.

This invention also provides a method of screening for an agent that increases or inhibits programmed cell death. The method involves contacting a cell (e.g. nematode cell, invertebrate cell, mammalian cell, human cell, etc.) comprising a BTF3 nucleic acid or polypeptide with a test agent; and detecting a change in the expression level or activity of BTF3 wherein an increase in BTF3 expression or activity, as compared to a control, indicates that said agent inhibits programmed cell death, while a decrease in BTF3 expression or activity, as compared to a control, indicates that said agent increases programmed cell death. In certain embodiments, the method involves measuring the expression level of a BTF3 gene in said cell (e.g. by measuring BTF3 mRNA, BTF3 polypeptide, etc.). In certain embodiments, the detecting comprises measuring/detecting the death of the cell. In certain embodiments, the detecting comprises detecting a BTF3 mRNA or cDNA and/or a BTF3 polypeptide, and/or BTF3 polypeptide activity. In certain embodiments, the detecting can comprise detecting BTF3 interaction with a caspase (e.g. using a two hybrid system, a binding assay, etc.). The level of BTF3 mRNA (or a nucleic acid derived therefrom) can be measured is measured by hybridizing said mRNA to a probe that specifically hybridizes to a BTF3 nucleic acid (e.g. in a Northern blot, a Southern blot using DNA derived from the BTF3 RNA, an array hybridization, an affinity chromatography, an in situ hybridization, etc.). In certain embodiments, the probe is a member of a plurality of probes that forms an array of probes. The level of BTF3 mRNA can also be measured using a nucleic acid amplification reaction (e.g. PCR).

The BTF3 polypeptide can be detected by a variety of methods known to those of skill in the art (e.g. capillary electrophoresis, a Western blot, mass spectroscopy, ELISA, immunochromatography, immunohistochemistry, etc.). The cell can be a cell cultured ex vivo. Or can be a cell present in a tissue, organ, or animal. In certain embodiments, the test agent is not an antibody, and/or not a nucleic acid and/or not a protein. Preferred test agents include small organic molecules. The method can further involve recording test agents that alter expression of the BTF3 nucleic acid or the BTF3 protein in a database of modulators of programmed cell death.

This invention also provides a method of prescreening for an agent that agent that modulates programmed cell death. The method involves i) contacting a BTF3 nucleic acid or a BTF3 polypeptide with a test agent; and ii) detecting specific binding of the test agent to said BTF3 nucleic acid or BTF3 polypeptide wherein specific binding of the test agent to the nucleic acid or to said polypeptide indicates that the agent is likely to modulate programmed cell death. The contacting can be in a cell (e.g. a nematode cell, a mammalian cell, a human cell, etc.). The method can further involve recording test agents that specifically bind to the nucleic acid or to polypeptide in a database of candidate agents that alter programmed cell death. In certain embodiments, the test agent is not an antibody and/or not a protein, and/or not a nucleic acid. Preferred test agents include small organic molecules. The detecting can comprise comprises detecting specific binding of said test agent to said nucleic acid (e.g. via a Northern blot, a Southern blot using DNA derived from an BTF3 RNA, an array hybridization, an affinity chromatography, and an in situ hybridization, etc.). The detecting can comprise detecting specific binding of the test agent to the BTF3 nuclear hormone receptor (e.g. capillary electrophoresis, a Western blot, mass spectroscopy, ELISA, immunochromatography, 2-hybrid assay, immunohistochemistry, etc.). The test agent can be contacted directly to the BTF3 nucleic acid and/or to the BTF3 polypeptide, to a cell containing the BTF3 polypeptide or BTF3 nucleic acid, or to an animal. In certain embodiments, the detecting can comprise detecting specific binding of the test agent to a caspase cleavage site of BTF3 and/or to a casein kinase II phosphorylation site of BTF3.

### DEFINITIONS

The terms “polypeptide”, “peptide” and “protein” are used interchangably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 14119), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2. times. SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1.times. SSC at 45° C. for 15 minutes. An example of a low stringency wash for a duplex of, e.g., more than 100 nucleotides, is 4× to 6×SSC at 40° C. for 15 minutes.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.go- v/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intranet, data or databases stored in specialized hardware (e.g. in microchips), and the like.

The phrase “down-regulation of BTF3” refers to inhibition of BTF3 (e.g. ceBTF3 and its homologues) expression and/or activity. Inhibition of expression can involve inhibition of transcription and/or translation and/or subsequent BTF3 protein processing (e.g. glycosylation, etc.). Inhibition of expression can also involve disruption of the regulation of BTF3 transcription. Inhibition of BTF3 activity includes, but is not limited to BTF3 antagonism, binding of BTF3 binding sites, inhibition of the interaction between BTF3 and a caspase, and the like. Conversely, the phrase “upregulation of BTF3” refers to an increase in BTF3 (e.g. ce-BTF3 and its homologues) expression and/or activity. In preferred embodiments, the inhibition or increase is as compared to a control (e.g. a wild-type cell, a cell of the same type as the test cell, but contacted with a different amount (or no) test agent, etc.). The inhibition or increase is preferably at least a 1.2-fold difference, preferably a 1.5-fold difference, more preferably at least a 2-fold difference, and most preferably at least a 4-fold, 5-fold or even 10-fold difference from the control.

The terms “isolated” “purified” or “biologically pure” refer to material which is substantially or essentially free from one or more components that normally accompany it as found in its native state. With respect to nucleic acids and/or polypeptides the term can refer to nucleic acids or polypeptides that are no longer flanked by the sequences typically flanking them in nature. Isolated nucleic acids and polypeptides can thus include polypeptides and nucleic acids that are transfected back into a cell and may therefore be found again in a typical biological mileau.

### BRIEF DESCRIPTION OF THE DRAWINGS

FIG. 1 shows the amino acid sequence (SEQ ID NO: 1) and putative domains of ce-BTF3. The predicted amino acid sequence of ce-BTF3 is 161 amino acids long, and we have identified a number of putative protein association and modification sites within ce-BTF3. The putative caspase recruitment domain (CARD) is located throughout the protein (aa 1-161). There are a number of putative caspase cleavage sites throughout the protein, and are denoted with boxes. There is one putative cathepsin D cleavage site that is italicized. There are two putative casein kinase II phosphorylation sites that are highlighted in bold letters.

FIGS. 2A and 2B show a putative CARD region in ce-BTF3 through comparison with other proteins containing CARD regions. FIG. 2A (SEQ ID NOS:2-19) shows a protein sequence comparison using the BLOCK Maker algorithm (BLOCKS) of ce-BTF3 with proteins identified as having CARD regions. The identical or conserved amino acids are shown shaded. FIG. 2B shows a protein sequence comparison (SEQ ID NOS:20-31) using clustal W (GenomeNet) of ce-BTF3 with known CARD proteins. Identical or conserved amino acids are shaded.

FIG. 3 shows the homology between ce-BTF3 and human BTF3. ce-BTF3 is compared to the human homologue of BTF using Blast 2 sequence comparison. ce-BTF3 is 63% identical and 75% similar to hu-BTF3 over a vast majority of the protein (ce-BTF3 is 161 amino acids long). + symbols indicated amino acid differences tat are considered conservative changes. −symbols indicate gaps introduced into the protein by the program to optimize the comparison. Sequence 1: huBTF3 (SEQ ID NO:32); Sequence 2: ceBTF3 (SEQ ID NO:33). Identity (SEQ ID NO:34). Identitiers=99/155 (63%, Positives=119/155 (75%), Gaps=10/155 (6%).

FIG. 4 shows that overexpression of ce-BTF3 decreases cell corpses in C. elegans embryos.

FIG. 5 illustrates inactivation of ce-BTF3 via RNAi increases cell corpses in C. elegans embryos.

FIG. 6 illustrates morphological phenotypes associated with ce-BTF3 RNAi. Wild-type young adults were soaked in double-stranded ce-BTF3 RNA to remove endogenous ce-BTF3 activity. Embryos, larvae and adult progeny were collected 24-48 hours post-soaking and scored for morphological phenotypes. Panel A) Example of embryonic phenotype associated with ce-BTf3 RNAi. This embryo is believed to have progressed past two-fold stage, with the pharynx present, as well as gut granules. Panel B) Example of LI larval phenotype associated with ce-BTF3 RNAi. The tail region of this larva is underdeveloped and uncoordinated. The pharynx appears normal, but the head region is misshapen and contains cell corpses. Panel C) Example of L2 larval phenotype associated with ce-BTF3 RNAi. Head region contains large vacuole where cell corpses are often found in earlier stages of development associated with ce-BTF3 RNAi. The pharynx runs beneath the vacuole. Panel D) Example of adult gonad phenotype associated with ce-BTF3 RNAi. Germ cells throughout the gonad arm are destroyed and the large, vacuolated regions develop. In some cases the vacuolization is proximal to the vulva, and in other it is distal.

FIG. 7 illustrates expression patterns of ce-BTF3 in adult C. elegans. Wild-type adult worms were fixed and stained with a primary antibody that recognizes ceBTF3. This primary antibody was bound by a secondary antibody conjugated to rhodamine, and fluorescence was observed as an indication of ce-BTF3 presence. Panel A). The head region of an adult worm containing the neurons of the nerve ring. There are tens of neurons within the nerve ring, and the arrows indicate regions that contain several neurons. Panel B) Fluorescent signal indicating the presence of ce-BTF3 in the neurons of the nerve ring. The pattern of staining (positive staining surrounding unstained nuclei) indicates that ce-BTF3 is expressed in the cytoplasm of these cells, and not in the nucleus. Panel C) The tail region of an adult worm containing a portion of the ventral nerve cord. Most of the nerve cord in this figure was destroyed during fixation, but a portion of the cord near the tail remained intact. Arrows indicate three ventral nerve cord neurons still present. Panel D) Fluorescent signal indicating the presence of ce-BTF3 in the neurons of the ventral nerve cord. As with the cells of the nerve ring, ce-BTF3 appears to be expressed in the cytoplasm of these cells. Panel E) The gonad region of an adult worm. This region of the gonad arm is proximal to the vulva and contains a large number of germ cells. F) Fluorescent signal indicating the presence of ce-BTF3 in the germ cells of the gonad. The staining pattern indicates ce-BTF3 expression in the cytoplasm of this region.

FIG. 8 show the appearance of cell corpses in ced-3 mutant worms exposed to ce-BTF3 RNAi. ced-3 mutant young adults were soaked in double-stranded ce-BTF3 RNA to remove endogenous ce-BTF3 activity. Embryonic, larval and adult progeny were scored for the presence of cell corpses. Panel A) Embryo at pretzel stage of development. Cell corpses are present in the region of the worm that eventually produces the ventral nerve cord. Panel B) Head region of an adult worm. Cell corpses are present in the region of the head near the pharynx where the neurons of the nerve ring are found. Panel C) The ventral nerve cord of an adult worm. Cell corpses are present throughout the ventral nerve cord. Panel D) The gonad arm of an adult worm. Cell corpses are present in the region of the gonad proximal to the vulva.

FIG. 9 shows that ventral nerve cord neurons and nerve ring neurons are missing in C. elegans treated with ce-BTF3 RNAi. C. elegans young adults expressing GFP-markers for neurons were soaked in ce-BTF3 double-stranded RNA, and their progeny were scored for the presence or absence of the GFP-marked neurons. The strains of worms used contain integrated plasmids in which GFP expression is restricted to neurons. Panel A) GFP-fluorescence marking the ventral nerve cord of an untreated worm. The strong fluorescence to the left of the ventral nerve cord is the nerve ring in the head of the worm. Panel B) Missing ventral nerve cord cells in a worm treated with ce-BTF3 RNAi. While the fluorescence of the nerve ring remains strong, there are a number of ventral nerve cord neurons missing as indicated by a lack of fluorescence in this region. Panel C) GFP-fluorescence marking the neurons of the nerve ring of an untreated worm. There are tens of neurons in this region, and the strength of the fluorescence makes it difficult to identify individual cells. Panel D) Missing nerve ring neurons in a worm treated with ce-BTF3 RNAi. The dramatic decrease in the number of neurons in this region allows for the distinction of individual cells.

FIG. 10. shows that cell corpses derived from the intestine contain Ce-BTF3::GFP. Animals containing an integrated copy of Ce-BTF3::GFP expression vector in their genome were treated with double-stranded ce-BTF3 RNA, and their progeny were scored for the presence of cell corpses. Panel A) An example of an embryo containing several cell corpses derived from intestinal cells. The arrows indicate the position of the corpses. Panel B) GFP fluorescence identified in the same cell corpses as found in Panel A). The arrows indicate the location of the fluorescence present in the cells corpses identified in A).

FIG. 11 shows that high levels of ce-BTF3 prevent the death of cells normally fated to die via PCD in the pharynx of C. elegans. Embryos were collected from worms that contain an integrated plasmid expressing ce-BTF3 from a heat-shock promoter. These embryos were heat-shocked at 33° C. for 1 hour and allowed to develop to adulthood. Pharynx cells were counted to determine the presence or absence of cells normally fated to die via PCD during development. In this example, black arrows indicate cells that are normally present in the pharynx of an adult worm, and white arrows indicate the presence of cells normally fated to die during development. The average number of extra cells found in the pharynxes counted was 4.2+.backslash.−2.7 (n=10). The range of extra cells found in these pharynxes was from 0 to 8.

FIG. 12 shows that wild-type C. elegans treated with ce-BTF3 RNAi contain cell corpses in a region of the gonad arm normally free of cell corpses. Wild-type worms were treated with ce-BTF3 double-stranded RNA, and their progeny were scored for the presence of germ cell corpses in the gonad arm. Apoptosis typically occurs in germ cells of C. elegans at the midpoint of the gonad arm. This figure shows the presence of cell corpses in the region proximal to the vulva, a region that typically doesn't contain cell corpses. This observation was made several times in these studies, and was often seen in conjunction with large vacuolated region within the gonad (FIG. 6, panel D). We also observed cell corpses in the germ cells of male C. elegans, an observation that has never been reported before.

### DETAILED DESCRIPTION

This invention pertains to the discovery that Cenorhabditis elegans BTF3 (Ce-BTF3) plays a critical, negative-regulatory role in programmed cell death (PCD) in C. elegans. Overexpression of Ce-BTF3 leads to decreased programmed cell death, while inactivation of Ce-BTF3 leads to increased programmed cell death. We have identified a putative CARD region on Ce-BTF3 that we believe is involved in the regulation of apoptosis through direct (or indirect) association with the caspase CED-3, the protein thought to be required for all programmed cell death in C. elegans.

Ce-BTF3 is the first C. elegans protein identified as a downstream target of CED-3, and a novel negative regulator of programmed cell death. Because the core programmed cell death (apoptotic) pathway in C. elegans is conserved in human programmed cell death, data pertaining to the role of Ce-BTF3 in C. elegans programmed cell death applicable to the processes of programmed cell death in human cells. Given the similarity of the human BTF3 homologue to Ce-BTF3, it is likely that the former performs a similar cell death suppressing function to the latter. Therefore, human BTF3 a useful target for the development of therapeutics, diagnostics, and prognostics for human diseases associated with excessive or insufficient programmed cell death.

We have identified a novel, and previously unknown role for the protein BTF3 in the regulation of programmed cell death. This protein, and its corresponding gene, provide a new system for discovering novel procedures relevant to controlling programmed cell death-related diseases.

It is believed that BTF3 has not been previously shown to regulate programmed cell death in any organism. We have found that BTF3 in the nematode, Caenorhabditis elegans, negative regulates (represses) programmed cell death in a number of cell types, including neurons.

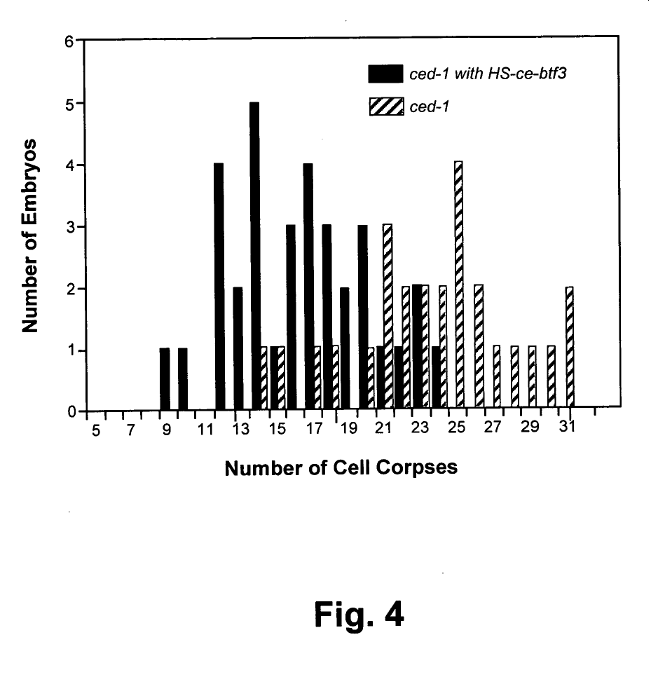

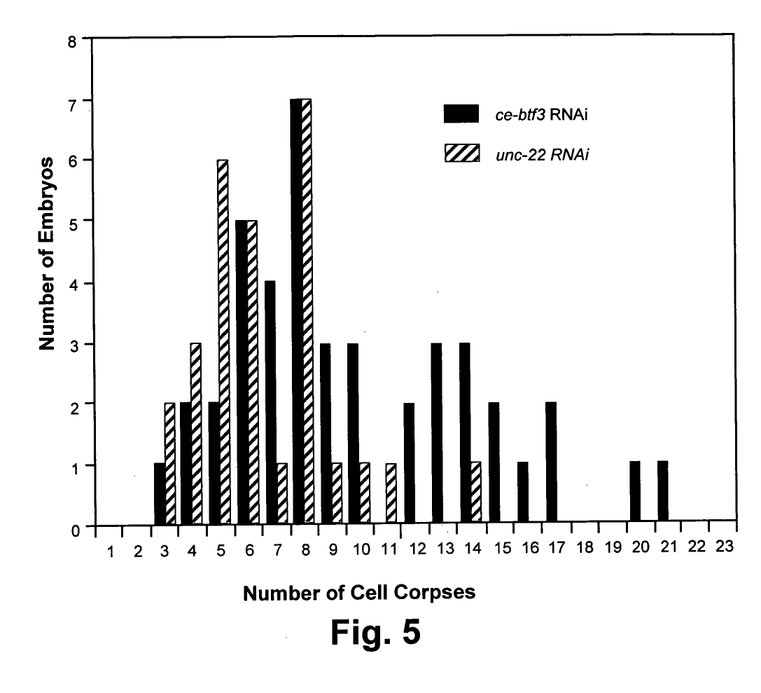

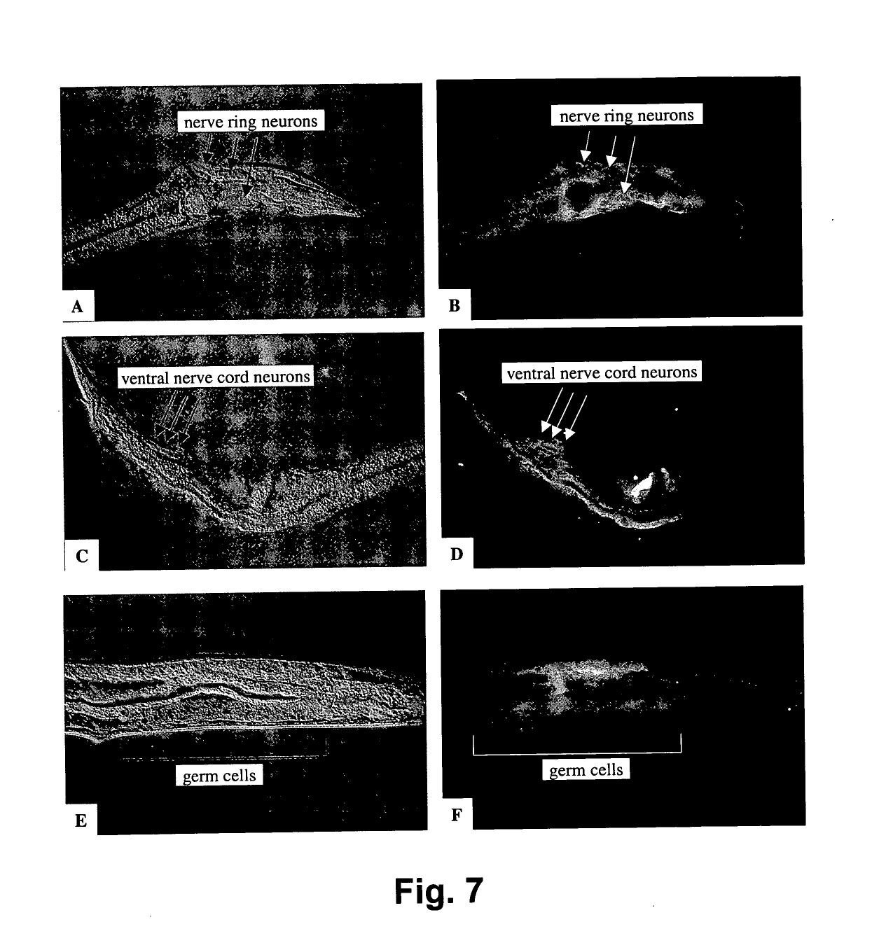

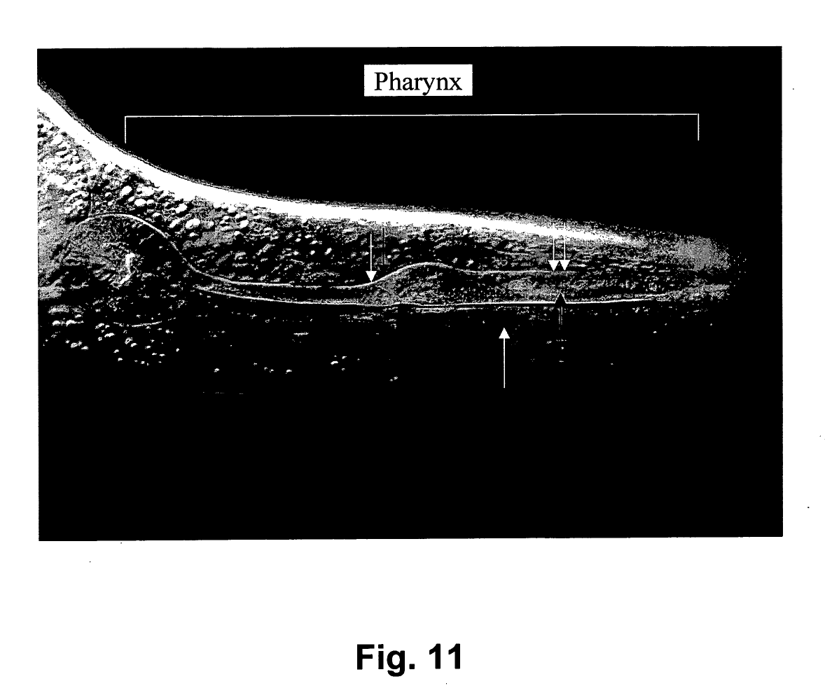

The following findings we have made demonstrate that BTF3 is both necessary and sufficient to repress programmed cell death: a) overexpression of the C. elegans BTF3 homologue (Ce-BTF3) in C. elegans embryos results in inhibition of developmentally programmed cell death (FIG. 4); This inhibition of programmed cell death leads to the presence of extra cells in the pharynx that are typically eliminated via PCD during development (FIG. 11)) reduction or removal of Ce-BTF3 activity in C. elegans leads to a profound increase in programmed cell death and elimination of many cells that would normally survive in the animal (FIG. 5) high levels of BTF3 expression (as detected by antibody staining) are seen in (but not limited to) neurons found in the head and tail regions of the animal, where most cell deaths occur during normal development. Ce-BTF3 is also detected throughout regions of the gonad arm of the animal, where a subset of germ cells are eliminated stochastically via apoptosis (FIG. 7).

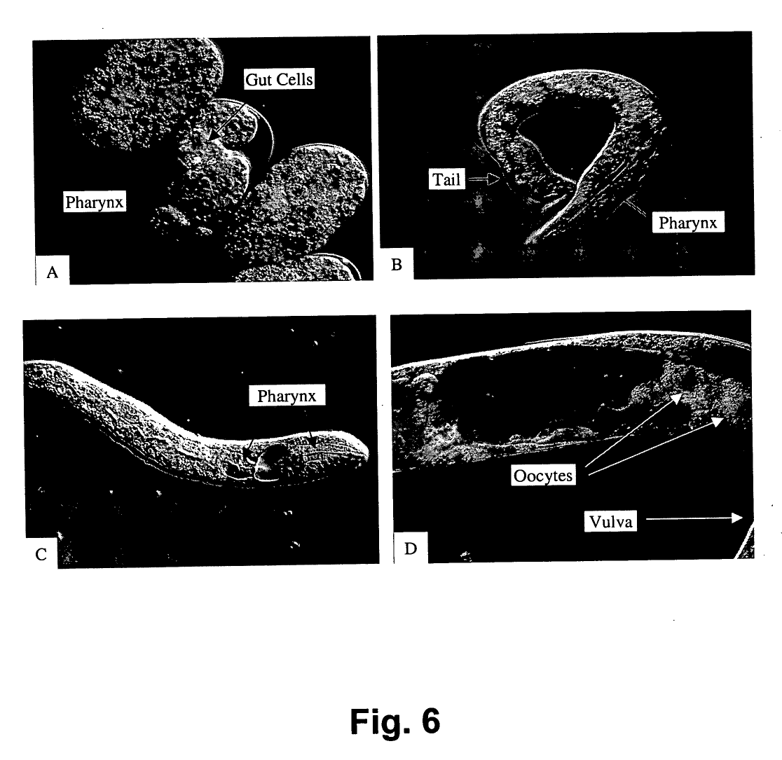

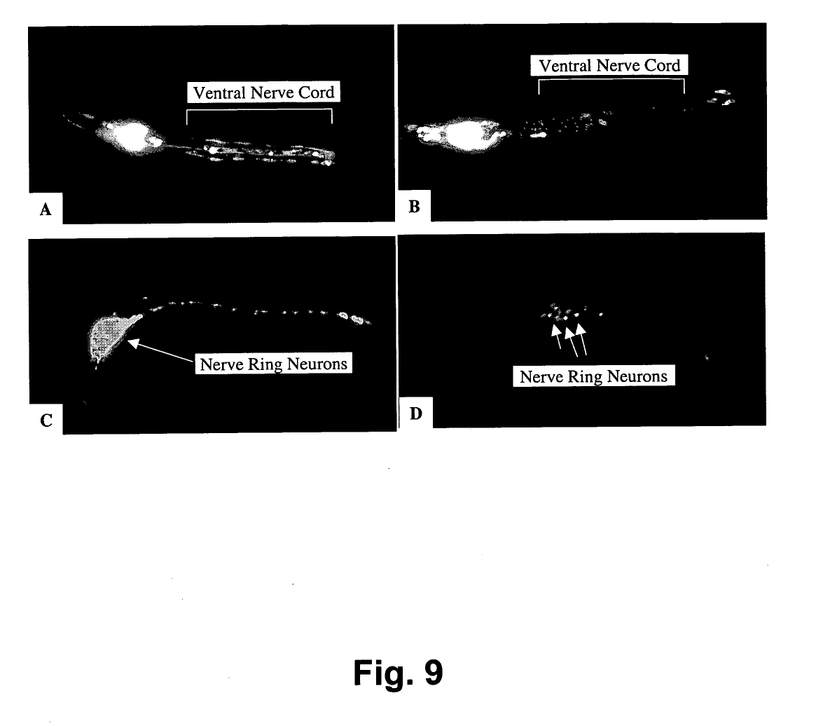

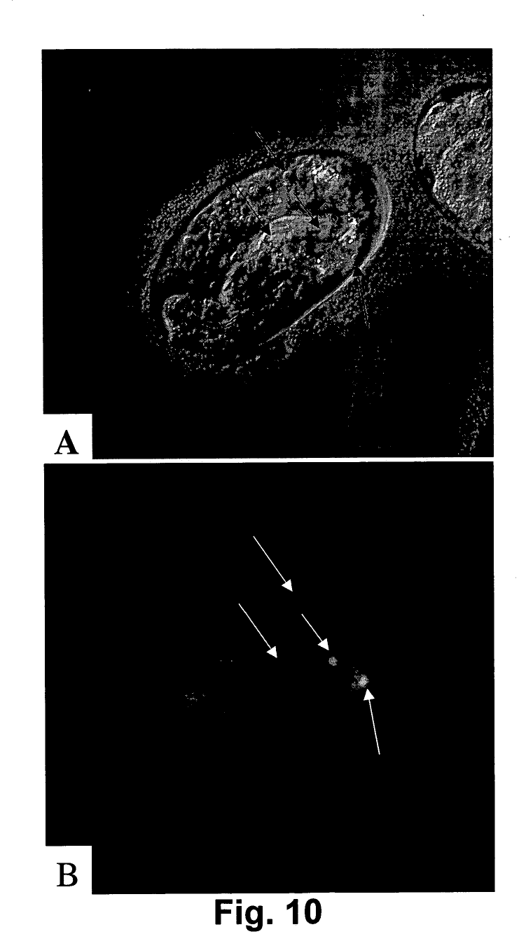

Animals in which Ce-BTF3 activity has been greatly diminished show phenotypes consistent with the occurrence of increased programmed cell death of neurons. These phenotypes include a large increase in morphologically typical cell corpses, large vacuolated regions in the head, uncoordinated movement and a shriveled tail region (FIG. 6). Also associated with decreased Ce-BTF3 activity are the elimination of ventral nerve cord neurons and the presence of cell corpses that contain Ce-BTF::green fluorescence protein (GFP), a marker for Ce-BTF3 expression in cells (FIG. 9 and FIG. 10 respectively). Thus, removal of BTF3 from C. elegans leads to a phenotype that resembles that of neurodegenerative diseases in humans, i.e. the inappropriate death of cells by an apoptotic process.

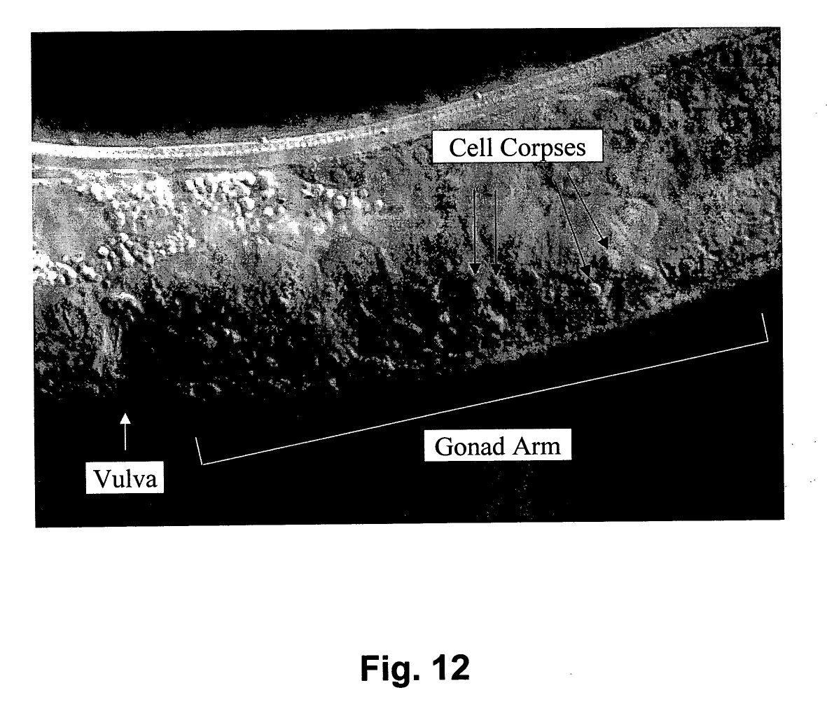

In addition to PCD associated with neurons, we have observed a role for ce-BTF3 in apoptosis of germ cells in C. elegans. Typically, germ cells in the animal undergo stochastic apoptosis in a specific region of the gonad arm and no where else. This region is found at the midpoint between the distal tip of the arm and the vulva. In animals lacking ce-BTF3 activity, apoptotic cell corpses were found proximal to the vulva in a region that normally doesn't contain cell corpses (FIG. 10). These observations, in conjunction with antibody staining that places ce-BTF3 in this region (FIG. 12), are consistent with the need for ce-BTF3 activity to prevent apoptosis in germ cells. Because the germ line of C. elegans is essentially immortal, the study of ce-BTF3 and proteins related to its activity in the prevention of PCD provides a valuable model for cancer development and the processes that allow preneoplastic cells to escape PCD control and become tumorgenic.

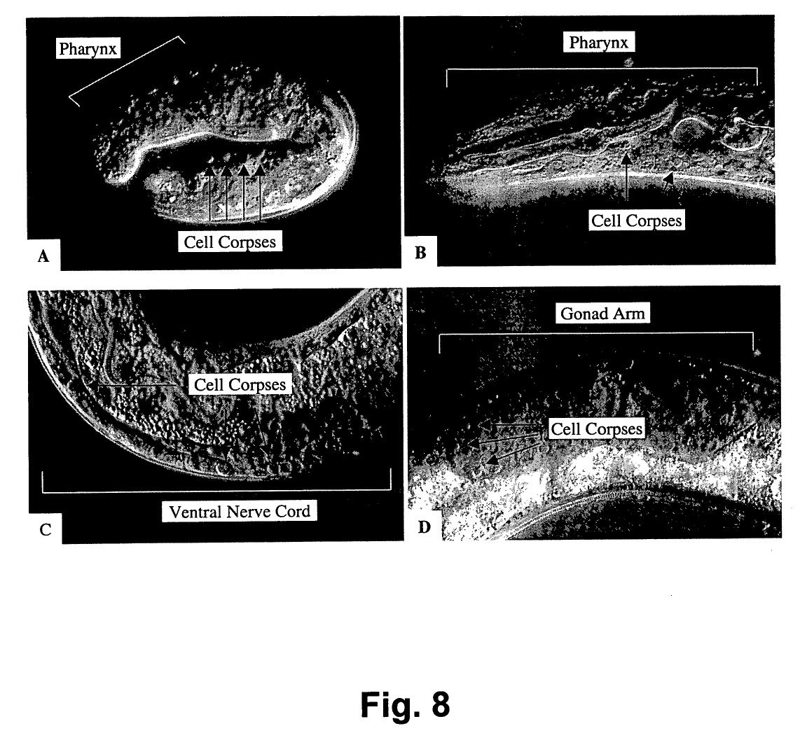

We have found that some of the excess programmed cell death observed in C. elegans embryos lacking BTF3 function is independent of the CED-3 caspase, while other cell deaths appear to be CED-3-dependent (FIG. 8). CED-3 is essential for virtually all cell death in normal animals. Although many cells undergo this caspase-independent cell death in BTF3-depleted animals, other cells are unaffected and remain viable and healthy well after the burst of cell death has occurred. As with wild-type animals, there is evidence in ced-3-mutant C. elegans that cell deaths occur in both the nerve ring and ventral nerve cord (FIG. 8). We have also observed CED-3-independent apoptosis in the germline (FIG. 8). These results suggest that BTF3 acts downstream of caspase function to maintain the viability of cells.

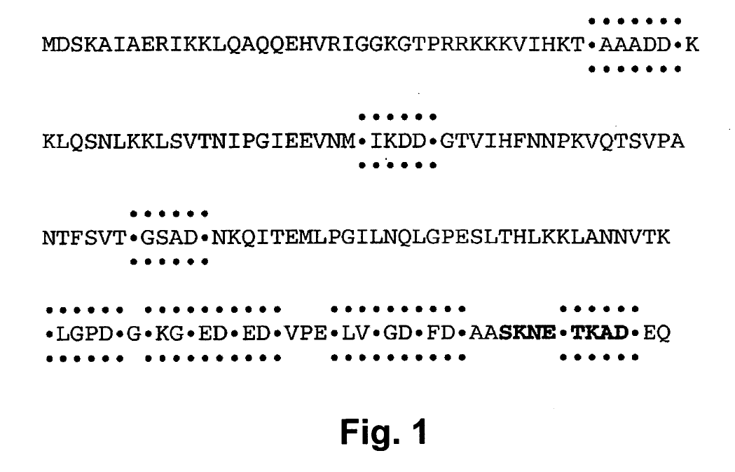

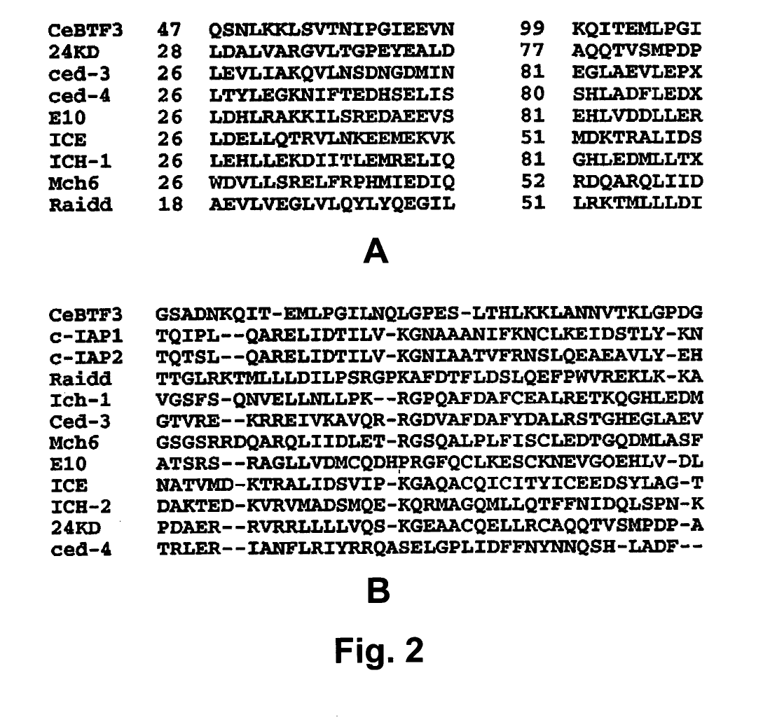

We have identified a number of putative protein association domains and modification sites that may affect the activities of Ce-BTF3 and allow it to perform its programmed cell death-suppressing function. These domains include a potential caspase recruitment domain (CARD) (FIG. 2), a number of caspase cleavage sites, cathepsin D cleavage sites and casein kinase II phosphorylation sites (FIG. 1). Together the genetic and structural observations suggest that Ce-BTF3 is a (possibly direct) downstream target of CED-3 activity and, as such, plays a crucial role in the initiation and/or implementation of programmed cell death, particularly in neurons. Ce-BTF3 appears to regulate programmed cell death negatively in cells until inactivated by CED-3, which promotes programmed cell death of that cell. Thus, BTF3 is not only a novel repressor of programmed cell death, but, is also the likely first target of CED-3 in programmed cell death. There is precedent for cleavage of hu-BTF3 by a caspase. BTF3 isolated from Jurkat T cells was found to be cleaved by caspase 3 in vitro (Thiede et al. (2001) J. Biol. Chem., 276: 26044-26050).

Some major advantages to identifying regulators of programmed cell death in C. elegans are the facts that much of the core programmed cell death pathway was first discovered and characterized in C. elegans, and that homologous proteins are known to perform the same functions in programmed cell death in humans. More specifically, the caspase CED-3 has been identified as the key effector of programmed cell death in C. elegans, and its human homologues play a similar role in human programmed cell death. The discovery that the CED-3 caspase activates cell death in C. elegans has led to the development of a new class of pharmaceuticals, namely caspase inhibitors, which are being tested in humans in an effort to prevent the progress of neurodegenerative diseases.

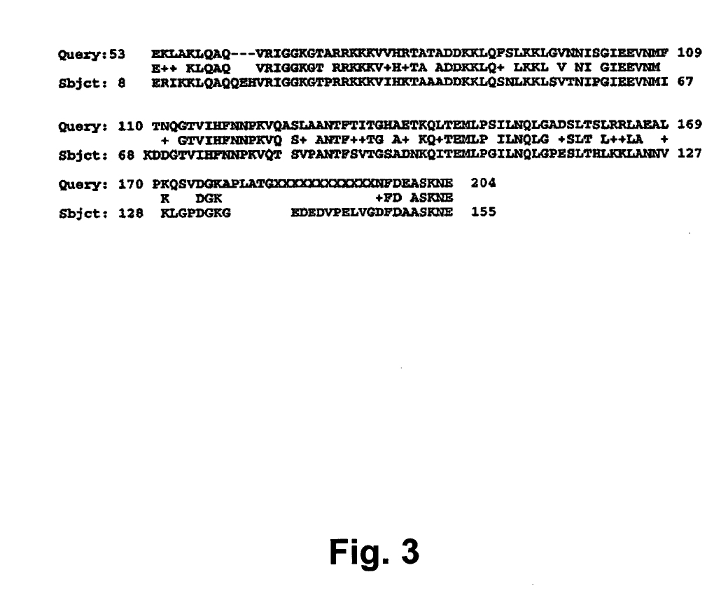

Humans express a homologue of Ce-BTF3 that is 60% identical and 70% similar to Ce-BTF3 (FIG. 3). Given its close similarity to the nematode protein, it is very likely that the human protein suppresses programmed cell death in humans. The characterization of the human homologue of Ce-BTF3 as a negative regulator of programmed cell death will provide a target for clinical treatments of human diseases associated with programmed cell death as well as a source of new reagents for the diagnosis and prognosis of such diseases.

Homology searches also indicate that plants contain apparent BTF3 proteins (e.g., in A. thaliana and curl leaf tobacco). Such processes as abscission, formation of xylem, and resistance to pathogens in plants are dependent on programmed cell death. Mounting evidence suggests that programmed cell death in plants uses similar control machinery as in animals. Therefore, methods that alter BTF3 activity in plants may prove of agricultural value.

I. Uses of BTF3

The gene encoding BTF3 is a useful gene to test in the full range of programmed cell death -related diseases. For example, mutations that inactivate the gene are expected to be responsible for certain neurodegenerative diseases, and mutations that hyperactivate or result in its expression in inappropriate cells result in tumors and cancer since cells in which programmed cell death has been inactivated are more likely to become tumorgenic. Methods that inactivate BTF3 function in tumor cells are expected to cause the selective death of cancer cells and elimination of the disease, regardless of its underlying cause. Similarly, treatments that inactivate BTF3 in cells infected with viruses or other pathogens, could prevent viral infection. Activation of BTF3 in neurons could prevent them from dying in neurodegenerative disease, regardless of the underlying cause.

The identification of BTF3 as a suppressor of cell death in C. elegans provides methods for identification of additional genes that act in the control of programmed cell death, using this animal as a model system. For example, isolation of genetic enhancer and suppressor mutations by either conventional genetic methods or reverse genetics methods (e.g., by RNA-mediated inactivation, or RNAi) could identify other factors that either promote programmed cell death or repress it, and thereby provide additional prognostics or diagnostics, or additional targets for drugs that might interfere with programmed cell death-related disease.

A) Down-Regulation of BTF3 in Cancer Therapy.

The treatments of cancer using nonsurgical methods generally consist of radiation therapy and cancer chemotherapy. Both therapies destroy rapidly proliferating cells through the generation of large amounts of mutation in the cell's genome. While these therapies are successful at eliminating cancer cells, they are also nonspecific, in that they eliminate proliferating healthy cells also. The killing of healthy cells by radiation and chemotherapy results in many severe side-effects. Treatments that are targeted to eliminate only cancer cells are highly sought after because such treatments would reduce or eliminate the severe side-effects of radiation and chemotherapy. One attractive target for cancer cell-specific elimination is programmed cell death (apoptosis), which eliminates cells with high levels of DNA damage and is therefore often disrupted in cancer cells. Therapies that could either bypass or rectify the defective aspect of programmed cell death in a cancer cell would facilitate the elimination of that cell through the reactivation of an apoptosis cascade, without eliminating or damaging the surrounding healthy cells. Such a therapy would be likely to better tolerated by a patient than convention chemotherapeutics or radiotherapy.

B) Upregulation of BTF3 to Inhibit Degenerative Diseases.

There exist many degenerative diseases (including such neurodegenerative diseases as ALS, Alzheimer's, Parkinson's, Multiple Sclerosis, Spinal Muscular Atrophy, etc.) and other pathological conditions (e.g., damage caused by stroke and heart disease) in which the pathology is the result of inappropriate programmed cell death. Treatments for such programmed cell death -related diseases are few and generally of little efficacy for the prevention or elimination of the disease. In addition, the diagnostic and prognostic tools available for most of these diseases are non-existent or very limited.

Some pharmaceuticals have been shown to be effective in slowing down the processes underlying degenerative diseases; however, these do not block the eventual development of the disease. Recently, transplants of stem cells contained in fetal tissue to the brains of Alzheimer's patients have been performed, but early results of these treatments are mixed, in that some patients may be regenerating lost neurons, while others are not.

A diagnostic or prognostic method that could quantify the levels of a protein that is predictive of the disregulation of programmed cell death will identify patients or conditions in which neurons are likely to undergo programmed cell death. Such a method could identify early states of neurodegenerative diseases, allowing for early intervention before the need for clinical presentation.

Furthermore, a therapy that abrogates the onset of programmed cell death in neurons by disrupting the negative-regulatory activities of an anti-programmed cell death protein could prevent that cell from being eliminated, thereby blocking the occurrence or progress of neurodegenerative disease.

The discovery of gene and protein functions in C. elegans can lead to rapid development of new medical methodologies. For example, the discovery that an initiator of apoptosis in this animal is a caspase enzyme led to the development of caspase inhibitors (Garcia-Calvo et al. (1998) J. Biol. Chem., 273: 32608-32613; Rasper et al. (1998) Cell Death and Differentiation, 5: 271-288). Caspase inhibitors are currently being used in clinical trials in humans in an effort to block neurodegenerative diseases, amply demonstrating that gene discovery in C. elegans can lead directly, and rapidly, to novel medical therapeutics. Thus, the strong conservation of structure and function in C. elegans genes makes this organism a powerful model system for discovering novel molecules which may prove to be relevant targets for clinical intervention in humans.

C) BTF3 Antibodies

The identification of Ce-BTF3 as an anti-programmed cell death protein allows us to develop diagnostic, prognostic, and therapeutic antibodies that could identify and treat patients with levels of BTF3 that make the cells resistant to programmed cell death. Such a reagent would indicate a disease state associated with resistance to programmed cell death, such as cancer. In addition, antibodies that block activity of BTF3 could make cancer cells once again susceptible to programmed cell death, thereby eliminating them from the patient.

D) Modulation of Apoptosis

The characterization of Ce-BTF3 as an anti-programmed cell death protein makes possible gene therapies that manipulate BTF3 activities for specific ends. Introducing wild-type BTF3 activity via a targeting expression vector into a cell susceptible to inappropriate programmed cell death, such as any of the many neurodegenerative diseases, could rescue the cell from programmed cell death. Conversely, introducing a dominant negative form of BTF3 into a cell resistant to programmed cell death due to high levels of BTF3, such as a cancer cell, can inactivate the endogenous BTF3, leading to programmed cell death of that cell. Manipulation of BTF3 activity in human cells via gene therapy provides a means for the preservation or elimination of cells identified as having aberrant programmed cell death activities.

The identification and characterization of the putative protein association and modification domains on Ce-BTF3 make it possible to alter these domains and identify their roles in the activity of Ce-BTF3 and, accordingly, their roles in the activities of homologous BTF3 proteins (e.g. hu-BTF3). The perturbation of the putative CARD region is expected to inactivate Ce-BTF3 (or hu-BTF3), making this domain a useful target for disruption in diseases associated with overactive Ce-BTF3 (e.g. cancer). Mutation of the numerous caspase cleavage sites and/or cathepsin D site could make Ce-BTF3 resistant to inactivation, and such a protein can be used in gene therapy to supply a constitutively active BTF3 to cells that need to be protected from aberrant programmed cell death, such as in neurodegenerative diseases. This type of protein could also contain a site that could allow for the inactivation of this protein when its anti-programmed cell death activity is no longer required in the cell. Mutation of the putative casein kinase II sites may either activate or inactivate Ce-BTF3, and a protein containing such mutations could be used accordingly in treatments that target human diseases associated with aberrant programmed cell death. Based on this information, pharmaceuticals could be developed that affect the above domains in the same ways as perturbation of these domains affects them.

The identification of mutations in Ce-BTF3 that correlate with the activation or inactivation of the protein makes it possible to identify homologous mutations in huBTF3 and correlate these mutations with disease. Such correlations would lead to the development of diagnostics and prognostics associated with the treatment of the diseases promoted by these mutations. As an example, if disruption of a caspase cleavage site in Ce-BTF3 leads to constitutive activation of the protein, then a similar mutation in hu-BTF3 could lead to its constitutive activation and subsequently to a disease state associated with the disruption of programmed cell death (e.g. cancer). Other mutations in Ce-BTF3 that upregulate or downregulate its activity could correlate with mutations in hu-BTF3 that are associated with neurodegenerative diseases, or any disease associated with aberrant programmed cell death activity. This information can be used to develop diagnostics that screen for such mutations in human cells, and prognostics that predict whether such mutations are likely to lead to the development of a disease, such as cancer or a neurodegenerative disease.

By genetically engineering the promoter region of Ce-BTF3 or hu-BTF3 into vectors that produce cytotoxic agents upon transcriptional activation, it is be possible to develop therapeutics that kill cells in which endogenous BTF3 is overproduced. Because the BTF3-promoter region on the vector will be upregulated to the extent of the endogenous promoter for BTF3, cells that overproduce BTF3 will also produce a cytotoxic agent that will kill the cell. Cells that escape programmed cell death through BTF3 overproduction would be eliminated before they can become tumorgenic. Such a vector can be targeted to precancerous lesions if they show an increased level of BTF3 production.

The identification of Ce-BTF3 as a target for inactivation during programmed cell death allows us to identify other proteins involved in the initiation and implementation of programmed cell death in C. elegans and, by homology, in humans. One possible association region may be the putative CARD region we have identified on CeBTF3. This region could be used to identify proteins that bind to Ce-BTF3 to either activate or inactivate the protein and affect programmed cell death. Such proteins would become new targets for understanding and manipulating programmed cell death in human cells. Different variations would be tested in this assay, using other regions of Ce-BTF3 or regions of hu-BTF3 screened with C. elegans or human libraries. Agents that interfere with BTF3 function could be designed by identifying compounds that interfere through the CARD and other domains that we have identified (see above).

Rapid genetic and biochemical techniques (e.g. microarray analysis) can be used to identify genes whose levels of transcription are affected by Ce-BTF3. Such genes are likely to play some role in programmed cell death, and their human homologues will immediately become the focus of studies on human programmed cell death. If Ce-BTF3 controls translational activities, genes whose transcripts are affected by Ce-BTF3 activity can be identified, and those genes can provide additional targets for medical intervention. In this way, the number of targets for the development of diagnostics, prognostics, and therapeutics used to treat diseases associated with aberrant programmed cell death can be increased.

C. elegans can be used as a model organism for the identification of agents that suppress BTF3 anti-programmed cell death activity. Suppressor mutations that counteract Ce-BTF3 activity can be generated and identified quickly in standard genetic suppressor screens. The genes associated with these mutations will be identified and they, or their human homologues, could be used as diagnostics or therapeutics that treat cells in which BTF3 levels are abnormally high.

The identification of ce-BTF3 as playing a role in preventing germ cell apoptosis provides a valuable model system for the development of preneoplastic cells into cancer cells. To become immortal, cancer cells have to prevent undergoing apoptosis in their preneoplastic state. The germ line of any organism is essentially immortal, and therefore is a useful model for identifying activities associated with immortality. The use of the C. elegans germ line for such studies is especially advantageous because of the genetic tractability of the organism as well as the existence of a completely sequenced genome. Characterizing the role of ce-BTF3 (and all proteins related to ce-BTF3 activity) in the prevention of apoptosis in the germ cells of C. elegans may provide useful insights into the proteins and activities responsible for the escape from apoptosis that cancer cells undergo during tumorgenesis in humans. Such identified proteins could be used as targets for diagnostics and therapeutics associated with the identification and treatment of cancer in humans.

By increasing or decreasing Ce-BTF3 activity in C. elegans, the animal can be used as a model for neuronal cell death in humans. Because the processes of neuronal cell death in C. elegans is likely highly conserved in human neurons, studies of C. elegans neurons in which BTF3 levels have been altered may reveal how human neurons die naturally and in a diseased state. This model could also be used to test therapeutics developed to treat aberrant programmed cell death in humans. Therapeutics that prevent programmed cell death in C. elegans cells in which BTF3 activity has been removed may have the same effect in humans, becoming an effective treatment for programmed cell death-related degenerative. Conversely, therapeutics that trigger programmed cell death in the presence of high levels of BTF3 could also be effective in the treatment of human diseases such as cancer.

Methods that inactivate BTF3 in humans, such as antisense RNA directed against hu-BTF3 are expected to abrogate the protein's function as an anti-programmed cell death protein. Such treatments can be used to trigger programmed cell death in those cells to eliminate detrimental cells, such as those that cause tumors.

Diseases associated with aberrant programmed cell death caused by abnormal BTF3 activity can readily be identified and similar diagnostics and therapeutics can be developed analogous to those described here for cancer and neurodegenerative disease.

Identifying Ce-BTF3 as a component of the programmed cell death pathway makes it possible to develop reagents and assays that can be used to study other aspects of the programmed cell death pathway in humans and other organisms. The recognition that Ce-BTF3 is a target of caspase activity, allows it to be used as a positive control for testing other potential caspases. Antibodies that recognize Ce-BTF3 can be used to coimmunoprecipitate proteins that associate with Ce-BTF3, providing insights into other proteins involved in programmed cell death. Many aspects of programmed cell death can be studied by developing reagents and assays based on the programmed cell death-repressive activity of BTF3.

The caspase cleavage sites of BTF3 provide a substrate for the study of other proteins involved in caspase cleavage, as can the cathespsin D cleavage sites provide a substrate for proteins involved in cathepsin D cleavage. Agents that bind to these sites are likely to be productive in interfering with or activating BTF3 function. Similarly, the casein kinase II phosphorylation sites may be used to develop agents that interfere with BTF3's function in programmed cell death. Any insights gained in the study of Ce-BTF3 and it putative association and modification sites may be used to generate reagents that would aid in the study of the processes associated with these sites.

All of the methods described above can also be applied toward the control of programmed cell death in plants. We believe the plant BTF3 proteins function to suppress programmed cell death in plants and methods that activate or inactivate BTF3 activity in plants are expected to prove useful for developing genetically improved strains or agents that improve survival or productivity of agriculturally important plants.

II. Assays for Modulators of BTF3 Expression.

As indicated above, in one aspect, this invention is premised, in part, on the discovery that BTF3 inhibits programmed cell death (e.g. the apoptotic cascade). Conversely, it is believed that downregulation of BTF3 will induce and/or increase apoptosis. Agents that downregulate BTF3 are expected to increase apoptosis, while agents that upregulate BTF3 are expected to inhibit programmed cell death.

Thus, in one embodiment, this invention provides methods of screening for agents that modulate BTF3 expression and/or activity. In certain preferred embodiments, the methods involve detecting the expression level and/or activity level of BTF3 genes or gene products (e.g. BTF3 mRNA or proteins) in the presence of the agent(s) in question. A reduced BTF3 expression level or activity level in the presence of the agent as compared to a negative control where the test agent is absent or at reduced concentration indicates that the agent downregulates BTF3 activity or expression. Conversely, increased BTF3 expression level or activity level in the presence of the agent as compared to a negative control where the test agent is absent or at reduced concentration indicates that the agent upregulates BTF3 activity or expression.

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from the BTF3 genes), level of translated protein, activity of translated protein, etc. Examples of such approaches are described below.

A) Nucleic-Acid Based Assays.

1) Target Molecules.

Changes in expression level can be detected by measuring changes in BTF3 genomic DNA or a nucleic acid derived from the genomic DNA (e.g., BTF3 m-RNA, reverse-transcribed cDNA, etc.). In order to measure the BTF3 expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA or a nucleic acid derived from an mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part L Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. (1987) Greene Publishing and Wiley-Interscience, New York).

Frequently, it is desirable to amplify the nucleic acid sample prior to assaying for expression level. Methods of amplifying nucleic acids are well known to those of skill in the art and include, but are not limited to polymerase chain reaction (PCR, see. e.g., Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of BTF3 in a sample, the nucleic acid sample is one in which the concentration of the BTF3 mRNA transcript(s), or the concentration of the nucleic acids derived from the BTF3 mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the BTF3-containing nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-Based Assays.

Using the known sequence of BTF3 (see sequence listing) detecting and/or quantifying the BTF3 transcript(s) can be routinely accomplished using nucleic acid hybridization techniques (see, e.g., Sambrook et al. supra). For example, one method for evaluating the presence, absence, or quantity of BTF3 genomic DNA or reverse-transcribed cDNA involves a “Southern Blot”. In a Southern Blot, the DNA typically fragmented and separated on an electrophoretic gel, is hybridized to a probe specific for BTF3. Comparison of the intensity of the hybridization signal from the BTF3 probe with a “control” probe (e.g. a probe for a “housekeeping gene) provides an estimate of the relative expression level of the target nucleic acid.

Alternatively, the BTF3 mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target BTF3 mRNA. Appropriate controls (e.g. probes to housekeeping genes) provide a reference for evaluating relative expression level.

An alternative means for determining the BTF3 expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure BTF3 expression (transcription) level. In such amplification-based assays, the target nucleic acid sequences (i.e., BTF3) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., BTF3 mRNA) in the original sample. Comparison to appropriate (e.g. healthy tissue or cells unexposed to the test agent) controls provides a measure of the BTF3 transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA (or other) standard. Detailed protocols for quantitative PCR are provided in PCR Protocols, A Guide to Methods and Applications, Innis et al. (1990) Academic Press, Inc. N.Y. The nucleic acid sequence(s) for BTF3 provided herein are sufficient to enable one of skill to routinely select primers to amplify any portion of the gene.

4) Hybridization Formats and Optimization of Hybridization Conditions.

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.). This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with .sup.3H, .sup.1251, .sup.35S, .sup.14C., or .sup.32P-labelled probes or the like. Other labels include ligands that bind to labeled antibodies, fluorophores, chemi-luminescent agents, enzymes, and antibodies, which can serve as specific binding pair members for a labeled ligand.

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25.times. SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-i DNA, etc.) during the hybridization to reduce non-specific binding. The use of blocking agents in hybridization is well known to those of skill in the art (see, e.g., Chapter 8 in P. Tijssen, supra.).

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of BTF3 expression levels can be full length or less than the full length of the BTF3 mRNA. Shorter probes are empirically tested for specificity. Preferred probes are sufficiently long so as to specifically hybridize with the BTF3 target nucleic acid(s) under stringent conditions. The preferred size range is from about 20 bases to the length of the BTF3 mRNA, more preferably from about 30 bases to the length of the BTF3 mRNA, and most preferably from about 40 bases to the length of the BTF3 mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescent molecules.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye, which is unconjugated and characterized in an arbitrary solvent.

Detectable signal can also be provided by chemiluminescent and bioluminescent sources. Chemiluminescent sources include a compound, which becomes electronically excited by a chemical reaction and can then emit light, which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-On Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 20132016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays—Polypeptide Expression.

1) Assay Formats.

In addition to, or in alternative to, the detection of BTF3 nucleic acid expression level(s), alterations in expression of BTF3 and/or activity of BTF3 can be detected and/or quantified by detecting and/or quantifying the amount and/or activity of translated BTF3 polypeptide or fragments thereof.

2) Detection of Expressed Protein.

The polypeptide(s) encoded by the BTF3 gene(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the BTF3 polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel clectrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In preferred embodiments, the BTF3 polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (BTF3 polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent /polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (BTF3 polypeptide) present in the sample is measured indirectly by measuring the amount of an added (exogenous) analyte displaced (or competed away) from a capture agent (antibody) by the analyte present in the sample. For example, in one competitive assay, a known amount of, in this case, labeled BTF3 polypeptide is added to the sample and the sample is then contacted with a capture agent. The amount of labeled polypeptide bound to the antibody is inversely proportional to the concentration of target BTF3 polypeptide present in the sample.

In one particularly preferred embodiment, the antibody is immobilized on a solid substrate. The amount of target polypeptide bound to the antibody may be determined either by measuring the amount of target polypeptide present in a polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind BTF3 polypeptide(s), either alone or in combination. In the case where the antibody that binds BTF3 polypeptide is not labeled, a different detectable marker, for example, an enzyme-labeled antibody capable of binding to the monoclonal antibody which binds the BTF3 polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds BTF3 polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration. Antibodies for use in the various immunoassays described herein, are commercially available or can be produced as described below.

4) Antibodies to BTF3 Polypeptides.

Either polyclonal or monoclonal antibodies (anti-BTF3 antibodies) may be used in the immunoassays of the invention described herein. Polyclonal antibodies are preferably raised by multiple injections (e.g. subcutaneous or intramuscular injections) of substantially pure polypeptides (BTF3 or fragments thereof) or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by BTF3.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant.

The general method used for production of hybridomas secreting mAbs is well known (Kohler and Milstein (1975) Nature, 256:495). Briefly, as described by Kohler and Milstein the technique comprises fusing an antibody-secreting cell (e.g. a splenocyte) with an immortalized cell (e.g. a myeloma cell). Hybridomas are then screened for production of antibodies that bind to BTF3 or a fragment thereof. Confirmation of specificity among mAb's can be accomplished using relatively routine screening techniques (such as the enzyme-linked immunosorbent assay, or “ELISA”, BiaCore, etc.) to determine the binding specificity and/or avidity of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 1010 nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold 1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V.sub.H and V.sub.L repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). BioTechnology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Polypeptide-Based Assays—Polypeptide Activity.

In addition to, or as an alternative to, the assays described above, it is also possible to assay for BTF3 activity. BTF3 activity can readily be determined by contacting a cell with an agent known to induce apoptosis. In one preferred embodiment, test agents (preferably test agents already shown to alter BTF3 activity or expression or to bind to BTF3 or a BTF3 gene product) are contacted to the cell and the ability of the apoptosis-inducing agent to induce apoptosis is assayed.

D) Pre-Screening for Agents That Bind BTF3 Nucleic Acids or Polypeptides.

In certain embodiments it is desired to pre-screen test agents for the ability to interact with (e.g. specifically bind to) an BTF3 nucleic acid or polypeptide. Specifically, binding test agents are more likely to interact with and thereby modulate BTF3 expression and/or activity. Thus, in some preferred embodiments, the test agent(s) are pre-screened for binding to BTF3 nucleic acids or to BTF3 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the BTF3 protein or protein fragment, or nucleic acid is immobilized and exposed to a test agent (which can be labeled), or alternatively, the test agent(s) are immobilized and exposed to an BTF3 protein (or fragment) or to an BTF3 nucleic acid or fragment thereof (which can be labeled). The immobilized moiety is then washed to remove any unbound material and the bound test agent or bound BTF3 nucleic acid or protein is detected (e.g. by detection of a label attached to the bound molecule). The amount of immobilized label is proportional to the degree of binding between the BTF3 protein or nucleic acid and the test agent.

III. High Throughput Screening for Agents That Modulate BTF3 Expression and/or Activity.

The assays of this invention are also amenable to “high-throughput” modalities. Conventionally, new chemical entities with useful properties (e.g., modulation of BTF3 activity or expression) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries for Modulators of BTF3 Expression and/or Activity

The likelihood of an assay identifying an modulator of BTF3 activity and/or expression is increased when the number and types of test agents used in the screening system is increased. Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 December 1991), encoded peptides (PCT Publication WO 93/20242, 14 October 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like). Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries for Agents for Modulators of BTF3 Expression and/or Activity.

Any of the assays for agents that modulate BTF3 expression or activity are amenable to high throughput screening. As described above likely modulators either inhibit expression of the gene product, or inhibit the activity of the expressed protein. Preferred assays thus detect inhibition of transcription (i.e., inhibition of mRNA production) by the test compound(s), inhibition of protein expression by the test compound(s), binding to the gene (e.g., gDNA, or cDNA) or gene product (e.g., mRNA or expressed protein) by the test compound(s) in the case of expression assays. High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

IV. Altering BTF3 or BTF3 Homologue Expression/Activity.

BTF3 expression can upregulated or inhibited using a wide variety of approaches known to those of skill in the art. For example, methods of ihibiting BTF3 expression include, but are not limited to antisense molecules, BTF3 specific ribozymes, BTF3 specific catalytic DNAs, intrabodies directed against BTF3 proteins, RNAi, gene therapy approaches that knock out BTF3, and small organic molecules that inhibit BTF3 expression/overexpression or block receptor that is required to induce BTF3 expression. BTF3 expression and/or activity can be up-regulated by introducing constructs expressing BTF3 into the cell (e.g. using gene therapy approaches) or upregulating endogenous expression of BTF3 (e.g. using agents identified in the screening assays of this invention). It will be appreciated that the methods used to alter BTF3 expression/activity can generally also be used to alter expression/activity of BTF3 homologues.

A) Antisense Approaches.

BTF3 gene expression can be downregulated or entirely inhibited by the use of antisense molecules. An “antisense sequence or antisense nucleic acid” is a nucleic acid that is complementary to the coding BTF3 mRNA nucleic acid sequence or a subsequence thereof. Binding of the antisense molecule to the BTF3 mRNA interferes with normal translation of the BTF3 polypeptide.

Thus, in accordance with preferred embodiments of this invention, preferred antisense molecules include oligonucleotides and oligonucleotide analogs that are hybridizable with BTF3 messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of BTF3 polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occurring portions. Thus, oligonucleotides may have altered sugar moieties or inter-sugar linkages. Exemplary among these are the phosphorothioate and other sulfur containing species which are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species which include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH.sub.3, F, OCH.sub.3, OCN, O(CH.sub.2)[n]NH.sub.2 or O(CH.sub.2)[n]CH.sub.3, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of BTF3 to inhibit the function of that RNA.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention may be conveniently and routinely made through the well-known technique of solid phase synthesis. Equipment for such synthesis is sold by several vendors, including Applied Biosystems. Any other means for such synthesis may also be employed, however, the actual synthesis of the oligonucleotides is well within the talents of the routineer. It is also will known to prepare other oligonucleotide such as phosphorothioates and alkylated derivatives.

Using the known sequence of the BTF3 gene/cDNA, appropriate and effective antisense oligonucleotide sequences can be readily determined.

B) Catalytic RNAs and DNAs

1) Ribozymes.

In another approach, BTF3 expression can be inhibited by the use of ribozymes. As used herein, “ribozymes” are include RNA molecules that contain anti-sense sequences for specific recognition, and an RNA-cleaving enzymatic activity. The catalytic strand cleaves a specific site in a target (BTF3) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes are provided which have the ability to inhibit BTF3 expression. Such ribozymes may be in the form of a “hammerhead” (for example, as described by Forster and Symons (1987) Cell 48: 211-220; Haseloff and Gerlach (1988) Nature 328: 596-600; Walbot and Bruening (1998) Nature 334: 196; Haseloff and Gerlach (1988) Nature 334: 585) or a “hairpin” (see, e.g. U.S. Pat. No. 5,254,678 and Hampel et al., European Patent Publication No. 013601257, published Mar. 26, 1990), and have the ability to specifically target, cleave and BTF3 nucleic acids.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO: 35). Suitable BTF3 of recognition or target sequences for hairpin ribozymes can be readily determined from the BTF3 sequence.

The sequence requirement at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an eight base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. The preferred ribozymes of this invention hybridize efficiently to target sequences at physiological temperatures, making them particularly well suited for use in vivo.

The ribozymes of this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Bimstiel (1989) EMBO J 8(12):3861-3866; Hempel et al. (1989) Biochem. 28: 4929-4933, etc.). After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89: 6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 27262730), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 014151731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W. H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo. Suitable transfector vectors for in vivo expression are discussed below. 2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 1014 DNAs containing 50 random nucleotides, successive rounds of selective amplification, enriched for individuals that best promote the Pb.sup.2+-dependent cleavage of a target ribonucleoside 3′-O—P bond embedded within an otherwise all-DNA sequence. By the fifth round, the population as a whole carried out this reaction at a rate of 0.2 min.sup.−1. Based on the sequence of 20 individuals isolated from this population, a simplified version of the catalytic domain that operates in an intermolecular context with a turnover rate of 1 min.sup.−1 (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10.sup.9 M.sup.−1min.sup.−1 under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to BTF3 mRNA thereby acting like a ribozyme.

C) Knocking out BTF3

In another approach, BTF3 can be inhibited/downregulated simply by “knocking out” the gene. Typically this is accomplished by disrupting the BTF3 gene, the promoter regulating the gene or sequences between the promoter and the gene. Such disruption can be specifically directed to BTF3 by homologous recombination where a “knockout construct” contains flanking sequences complementary to the domain to which the construct is targeted. Insertion of the knockout construct (e.g. into the BTF3 gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a poly-A sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

Suitable knockout constructs have been made and used to produce BTF3 knockout mice (see, e.g., Dorfman et al. (1996) Oncogene 13: 925-931). The knockout constructs can be delivered to cells in vivo using gene therapy delivery vehicles (e.g. retroviruses, liposomes, lipids, dendrimers, etc.) as described below. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

D) Intrabodies.

In still another embodiment, BTF3 expression/activity is inhibited by transfecting the subject cell(s) (e.g., cells of the vascular endothelium) with a nucleic acid construct that expresses an intrabody. An intrabody is an intracellular antibody, in this case, capable of recognizing and binding to a BTF3 polypeptide. The intrabody is expressed by an “antibody cassette”, containing a sufficient number of nucleotides coding for the portion of an antibody capable of binding to the target (BTF3 polypeptide) operably linked to a promoter that will permit expression of the antibody in the cell(s) of interest. The construct encoding the intrabody is delivered to the cell where the antibody is expressed intracellularly and binds to the target BTF3, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (VH) and light chain variable (V.sub.L) domains of an antibody which can be connected at the DNA level by an appropriate oligonucleotide as a bridge of the two variable domains, which on translation, form a single peptide (referred to as a single chain variable fragment, “sFv”) capable of binding to a target such as an BTF3 protein. The intrabody gene preferably does not encode an operable secretory sequence and thus the expressed antibody remains within the cell.

Anti-BTF3 antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for BTF3. Such screening methods are described above in some detail.

The antibody cassette is delivered to the cell by any of the known means. This discloses the use of a fusion protein comprising a target moiety and a binding moiety. The target moiety brings the vector to the cell, while the binding-moiety carries the antibody cassette. Other methods include, for example, Miller (1992) Nature 357: 455-460; Anderson (1992) Science 256: 808-813; Wu, et al. (1988) J. Biol. Chem. 263: 14621-14624. For example, a cassette containing these (anti-BTF3) antibody genes, such as the sFv gene, can be targeted to a particular cell by a number of techniques including, but not limited to the use of tissue-specific promoters, the use of tissue specific vectors, and the like. Methods of making and using intrabodies are described in detail in U.S. Pat. No. 6,004,940.

E) Small Organic Molecules.

In still another embodiment, BTF3 expression and/or BTF3 protein activity can be inhibited by the use of small organic molecules. Such molecules include, but are not limited to molecules that specifically bind to the DNA comprising the BTF3 promoter and/or coding region, molecules that bind to and complex with BTF3 mRNA, molecules that inhibit the signaling pathway that results in BTF3 upregulation, and molecules that bind to and/or compete with BTF3 polypeptides. Small organic molecules effective at inhibiting BTF3 expression can be identified with routine screening using the methods described herein.

The methods of inhibiting BTF3 expression described above are meant to be illustrative and not limiting. In view of the teachings provided herein, other methods of inhibiting BTF3 will be known to those of skill in the art.

F) Modes of Administration.

The mode of administration of the BTF3 blocking agent depends on the nature of the particular agent. Antisense molecules, catalytic RNAs (ribozymes), catalytic DNAs, small organic molecules, and other molecules (e.g. lipids, antibodies, etc.) used as BTF3 inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

1) Pharmaceutical Administration.

In order to carry out the methods of the invention, one or more inhibitors of BTF3 expression (e.g. ribozymes, antibodies, antisense molecules, small organic molecules, etc.) are administered to an individual to ameliorate one or more symptoms of atherosclerosis and/or rheumatoid arthritis. While this invention is described generally with reference to human subjects, veterinary applications are contemplated within the scope of this invention.

Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The BTF3 inhibitors and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The BTF3 inhibitors and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., atherosclerosis and/or associated conditions, and/or rheumatoid arthritis) in an amount sufficient to cure or at least partially arrest the disease and/or its symptoms (e.g. to reduce plaque formation, to reduce monocyte recruitment, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the BTF3 inhibitors are administered orally (e.g. via a tablet) or as an injectable in accordance with standard methods well known to those of skill in the art. In other preferred embodiments, the BTF3 inhibitors may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised. 2) Gene Therapy.

As indicated above, antisense molecules, catalytic RNAs (ribozymes), catalytic DNAs, and additionally, knockout constructs, and constructs encoding intrabodies can be delivered and transcribed and/or expressed in target cells (e.g. cancer cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Feigner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cometta et al. (1991) Hum. Gene Ther. 2: 215). For a review of gene therapy procedures, see, e.g., Anderson, Science (1992) 256: 808-813; Nabel and Feigner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 11491154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1: 13-26.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include, but are not limited to, herpes virus, lentivirus, and vaccinia virus.

V. Kits.

In still another embodiment, this invention provides kits for practice of the methods described herein. In certain embodiments the kits comprise a nucleic acid that hybridizes to a BTF3 nucleic acid and/or an antibody that specifically binds to a BTF3 polypeptide. Certain kits may comprise a vector that encodes a BTF3 polypeptide and/or a cell containing such a vector.

The kits may optionally include any reagents and/or apparatus to facilitate practice of the methods described herein. Such reagents include, but are not limited to buffers, instrumentation (e.g. bandpass filter), reagents for detecting a signal from a detectable label, transfection reagents, cell lines, vectors, and the like.

In addition, the kits may include instructional materials containing directions (i.e., protocols) for the practice of the methods of this invention. Preferred instructional materials provide protocols utilizing the kit contents for screening for agents that increase or decrease programmed cell death by increasing or decreasing BTF3 expression and/or activity, e.g., as described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

FIG. 1 shows the amino acid sequence and putative domains of ce-BTF3. The predicted amino acid sequence of ce-BTF3 is 161 amino acids long, and we have identified a number of putative protein association and modification sites within ce-BTF3. The putative caspase recruitment domain (CARD) is located throughout the protein (aa 1-161). There are a number of putative caspase cleavage sites throughout the protein, and are denoted with boxes. There is one putative cathepsin D cleavage site that is italicized. There are two putative casein kinase II phosphorylation sites that are highlighted in bold letters.

FIG. 2 shows a putative CARD region in ce-BTF3 through comparison with other proteins containing CARD regions. FIG. 2A shows a protein sequence comparison using the BLOCK Maker algorithm (BLOCKS) of ce-BTF3 with proteins identified as having CARD regions. The identical or conserved amino acids are shown shaded. FIG. 2B shows a protein sequence comparison using clustal W (GenomeNet) of ce-BTF3 with known CARD proteins. Identical or conserved amino acids are shaded.

FIG. 3 shows the homologue between ce-BTF3 and human BTF3. cdBTF3 is compared to the human homologue of BTF using Blast 2 sequence comparison. ce-BTF3 is 63% identical and 75% similar to hu-BTF3 over a vast majority of the protein (ce-BTF3 is 161 amino acids long). +symbols indicated amino acid differences tat are considered conservative changes. −symbols indicate gaps introduced into the protein by the program to optimize the comparison.

FIG. 4 shows that overexpression of ce-BTF3 decreases cell corpses in C. elegans embryos. C. elegans containing a ced-1 mutation were injected with a heat shock construct expressing ce-BTF3. A ced-1 mutation allows cell corpses to remain visible for long periods of time before being engulfed. The heat-shock construct produces ectopic expression of ce-BTF3 at 33° C. and resulting embryos were scored for cell corpses to the comma stage of development. ced-1 worms without the HS-ce-btf3 construct had 23.6.+−.4.5 cell corpses per embryo (n=26). ced-1 worms with HS-ce-btf3 had 16.4.+−.3.8 cell corpses per embryo (n=34).

FIG. 5 illustrates inactivation of ce-BTF3 via RNAi increases cell corpses in C. elegans embryos. ce-BTF3 activity was blocked in wild-type adult C. elegans using RNA interference (RNAi) and resulting embryos were scored at comma stage of cell corpses. AS a control, as separate population of wild-type adults were exposed to unc-22 RNAi to determine the effect of the RNAi process on cell corpse production. Worms treated with ce-btf3 RNA contained 10.±.4.4 cell corpses per embryo (n=42). Worms treated with unc-22 RNA contained 6.6.±.2.4 cell corpses per embryo (n=28).

FIG. 6 illustrates morphological phenotypes associated with ce-BTF3 RNAi. Wild-type young adults were soaked in double-stranded ce-BTF3 RNA to remove endogenous ce-BTF3 activity. Embryos, larvae, and adult progeny were collected 24-48 hours post-soaking and scored for morphological phenotypes. FIG. 6A shows an example of an embryonic phenotype associated with ce-BTF3 RNAi. This embryo is believed to have progressed past two-fold stage with the pharynx present, as well as gut granules. Panel B shows and example of L1 larval phenotype associated with ce-BF3 RNAi. The tail region of this larvae is underdeveloped and uncoordinated. The pharynx appears normal, but the head region is misshapen and contains cell corpses. Panel C shows an example of a L2 larval phenotype associated with ce-BTF3 RNAi. Head region contains large vacuole where cell corpses are often found in earlier stages of development associated with ce-bTF3 RNAi. The pharynx runs beneath the vacuole. Panel D shows an example of adult gonad phenotype associated with ce-BTF3 RNAi. Polarity of gonad arm in this adult is reversed, with mitotic germ cells located at the vulva, and developed oocytes located in the distal arm region (not shown). The larger-than-usual number of mitotic germ cells in this gonad arm is consistent with a tumorgenic cell phenotype. G

FIG. 7 shows the expression pattern of ce-BTF3 in adult C. elegans. Wild-type adult worms were injected with a plasmid containing a green fluorescent protein (GFP) expression construct under the control of 1 kb of the ce-BTF3 promoter region. Nonintegrated and integrated worm lines were isolated, and observed GFP fluorescence was interpreted to indicate the expression pattern of ce-BTF3 in C. elegans. Panel A shows ceBTF3 expression in the head region of an adult from a non-integrated line. Fluorescence is seen in numerous neurons found in the head region, including the nerve ring located near the posterior bulb of the pharynx. Fluorescence is also seen in the pharynx itself. Panel B shows ce-BTF3 expression n the head region of an adult from an integrated line. Fluorescence is seen in the neurons of the head as well as the ventral nerve cord, excretory cell, gut cells, and a number of muscle cells. Panel C shows ce-BTF3 expression in the tail region of an adult from an integrated line. There are a number of neurons located in the tail region of C. elegans, and ce-BTF3 expression is found in these neurons. Panel D shows ceBTF3 expression in the gonad of adult from an integrated line. Fluorescence is seen in the mitotic germ cells of the adult gonad.

FIG. 8 shows the appearance of cell corpses in ced-3 mutant worms exposed to ce-BTF3 RNAi. ced-3 mutant young adults were soaked in double stranded ceBTF3 RNA to remove endogenous ce-BTF3 activity. Embryonic and larval progeny were collected and scored for the presence of cell corpses. Panel A shows an embryo at comma stage scored for the presence of cell corpses. Arrows denote the presence of cell corpses located in the region of the embryo that produces a number of neurons, and ultimately gives rise to the pharynx and head of the animal. This region is the same as that which contains extra cell corpses in wild-type worms exposed to ce-BTF3 double-stranded RNA. Panel B shows LI larva scored for the presence of cell corpses. Arrows denote the presence of cell corpses in the pharynx region of the larva, a region that contains a number of neurons including those that constitute the nerve ring.

FIG. 9 shows that ventral nerve cord cells are missing in unc-119/GFP C. elegans treated with ce-BTF3 RNAi. Unc-119/GFP young adults were soaked in ce-BTF3 double-stranded RNA, and their progeny were scored for the presence or absence of ventral nerve cord cells. unc-119/GFP animals contain an integrated plasmid in which GFP expression is controlled by the unc-119 promoter. This construct causes fluorescence in a number of nerve cells throughout the body, including the ventral nerve cord. Panel A shows unc-119/GFP larva not treated with ce-BTF3 double-stranded RNA. The brightest fluorescence near the pharynx represents the nerve ring, while fluorescence occurring on the ventral side of the body represents the cells of the ventral nerve cord. Panel B shows unc119/GFP larva grated with the ce-BTF3 double-stranded RNA. Note the absence of a number of ventral nerve cord cells in the body of the animal.

FIG. 10 shows that cell corpses derived from the intestine contain Ce-BTF3::GFP. Animals containing an integrated copy of Ce-BTF3::GFP expression vector in their genome were treated with double-stranded Ce-btf3 RNA, and their progeny were scored for the presence of cell corpses. Panel A shows an example of an embryo containing several cell corpses derived from intestinal cells. The arrows indicate the position of the corpses. Panel B shows GFP fluorescence identified in the same cell corpses as found in FIG. 10A. The arrows indicate the location of the fluorescence present in the cells corpses identified in Panel A.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening for an agent that modulates programmed cell death, said method comprising:
a. providing a test cell containing a BTF3 molecule selected from the group consisting of: a BTF3 nucleic acid and a BTF3 polypeptide encoded by the nucleic acid;
b. contacting the BTF3 with a test agent; and
c. detecting a change in the activity of said BTF3 wherein an increase in BTF3 activity, as compared to a control, indicates that said agent inhibits programmed cell death, while a decrease in BTF3 activity, as compared to a control, indicates that said agent increases programmed cell death,
d. said BTF3 nucleic acid encoding and said BTF3 polypeptide comprising a sequence substantially identical to a sequence selected from the group consisting of: SEQ ID NO: 1, SEQ ID NO: 32, and SEQ ID NO: 3.

2. The method of claim 1, wherein said detecting comprises measuring the expression level of a BTF3 gene in said cell.

3. The method of claim 1, wherein said detecting comprises measuring the death of said cell.

4. The method of claim 1, wherein said cell is a mammalian cell.

5. The method of claim 1, wherein said cell is a nematode cell.

6. The method of claim 1, wherein said cell is a human cell.

7. The method of claim 1, wherein said detecting comprises detecting a BTF3 mRNA or cDNA.

8. The method of claim 1, wherein said detecting comprises detecting a BTF3 polypeptide.

9. The method of claim 1, wherein said detecting comprises measuring BTF3 polypeptide activity.

10. The method of claim 1, wherein said detecting comprises detecting BTF3 interaction with a caspase.

11. The method of claim 1, wherein the expression level of BTF3 is detected by measuring the level of BTF3 mRNA in said cell.

12. The method of claim 11, wherein said level of BTF3 mRNA is measured by hybridizing said mRNA to a probe that specifically hybridizes to a BTF3 nucleic acid.

13. The method of claim 12, wherein said hybridizing is according to a method selected from the group consisting of a Northern blot, a Southern blot using DNA derived from the BTF3 RNA, an array hybridization, an affinity chromatography, and an in situ hybridization.

14. The method of claim 12, wherein said probe is a member of a plurality of probes that forms an array of probes.

15. The method of claim 1, wherein said level of BTF3 mRNA is measured using a nucleic acid amplification reaction.

16. The method of claim 1, wherein said expression level of BTF3 is detected by determining the expression level of a BTF3 polypeptide in said biological sample.

17. The method of claim 16, wherein said detecting is via a method selected from the group consisting of capillary electrophoresis, a Western blot, mass spectroscopy, ELISA, immunochromatography, and immunohistochemistry.

18. The method of claim 1, wherein said cell is cultured ex vivo.

19. The method of claim 1, wherein said test agent is administered to an animal comprising a cell containing the BTF3 nucleic acid or the BTF3 protein.

20. The method of claim 1, wherein said test agent is an antibody.

21. The method of claim 1, wherein said test agent is a protein.

22. The method of claim 1, wherein said test agent is a small organic molecule.

23. The method of claim 1, further comprising recording test agents that alter expression of the BTF3 nucleic acid or the BTF3 protein in a database of modulators of programmed cell death.

24. A method of prescreening for an agent that modulates programmed cell death, said method comprising i) contacting at least one of a nucleic acid encoding BTF3 and a BTF3 polypeptide with a test agent; and ii) detecting specific binding of said test agent to said BTF3 nucleic acid or BTF3 polypeptide wherein specific binding of said test agent to said nucleic acid or to said polypeptide indicates that said agent is likely to modulate programmed cell death, wherein said BTF3 nucleic acid encoding and said BTF3 polypeptide comprising a sequence substantially identical to a sequence selected from the group consisting of: SEQ ID NO: 1, SEQ ID NO: 32, and SEQ ID NO: 3.

25. The method of claim 24, wherein said contacting is in a cell.

26. The method of claim 25, wherein said cell is a nematode cell.

27. The method of claim 25, wherein said cell is a mammalian cell.

28. The method of claim 25, wherein said cell is a human cell.

29. The method of claim 24, further comprising recording test agents that specifically bind to said nucleic acid or to said polypeptide in a database of candidate agents that alter programmed cell death.

30. The method of claim 24, wherein said test agent is an antibody.

31. The method of claim 24, wherein said test agent is a protein.

32. The method of claim 24, wherein said test agent is a nucleic acid.

33. The method of claim 24, wherein said test agent is a small organic molecule.

34. The method of claim 24, wherein said, wherein said detecting comprises detecting specific binding of said test agent to said nucleic acid.

35. The method of claim 34, wherein said binding is detected using a method selected from the group consisting of a Northern blot, a Southern blot using DNA derived from an BTF3 RNA, an array hybridization, an affinity chromatography, and an in situ hybridization.

36. The method of claim 24 wherein said detecting comprises detecting specific binding of said test agent to said polypeptide.

37. The method of claim 36, wherein said, wherein said detecting is via a method selected from the group consisting of capillary electrophoresis, a Western blot, mass spectroscopy, ELISA, immunochromatography, and immunohistochemistry.

38. The method of claim 24, wherein said test agent is contacted directly to an isolated BTF3 nucleic acid or BTF3 polypeptide.

39. The method of claim 24, wherein said, wherein said test agent is contacted to a cell containing the BTF3 polypeptide or BTF3 nucleic acid.

40. The method of claim 39, wherein said cell is cultured ex vivo.

41. The method of claim 24, wherein said, wherein said test agent is administered to an animal comprising a cell containing the BTF3 polypeptide or the BTF3 nucleic acid.

42. The method of claim 24, wherein said detecting comprises detecting specific binding of said agent to a caspase cleavage site of BTF3 as indicated in FIG. 1.

43. The method of claim 24, wherein said detecting comprises detecting specific binding of said agent to casein kinase II phosphorylation site of BTF3 as indicated in FIG. 1.


SEQUENCE LISTING (35 sequences)

SEQ ID NO: 1 - protein, 161 aa, Caenorhabditis elegans
MDSKAIAERIKKLQAQQEHVRIGGKGTPRRKKKVIHKTAAADDKKLQSNLKKLSVTNIPGIEEVNMIKDDGTVIHFNNPKVQTSVPANTFSVTGSADNKQITEMLPGILNQLGPESLTHLKKLANNVTKLGPDGKGEDEDVPELVGDFDAASKNETKADEQ

SEQ ID NO: 2 - protein, 19 aa, Caenorhabditis elegans
QSNLKKLSVTNIPGIEEVN

SEQ ID NO: 3 - protein, 10 aa, Caenorhabditis elegans
KQITEMLPGI

SEQ ID NO: 4 - protein, 19 aa, Caenorhabditis elegans
LDALVARGVLTGPEYEALD

SEQ ID NO: 5 - protein, 10 aa, Caenorhabditis elegans
AQQTVSMPDP

SEQ ID NO: 6 - protein, 19 aa, Caenorhabditis elegans
LEVLIAKQVLNSDNGDMIN

SEQ ID NO: 7 - protein, 10 aa, Caenorhabditis elegans
  misc_feature (10)..(10): X is any amino acid
EGLAEVLEPX

SEQ ID NO: 8 - protein, 19 aa, Caenorhabditis elegans
LTYLEGKNIFTEDHSELIS

SEQ ID NO: 9 - protein, 10 aa, Caenorhabditis elegans
  misc_feature (10)..(10): X is any amino acid.
SHLADFLEDX

SEQ ID NO: 10 - protein, 19 aa, Caenorhabditis elegans
LDHLRAKKILSREDAEEVS

SEQ ID NO: 11 - protein, 10 aa, Caenorhabditis elegans
EHLVDDLLER

SEQ ID NO: 12 - protein, 19 aa, Caenorhabditis elegans
LDELLQTRVLNKEEMEKVK

SEQ ID NO: 13 - protein, 10 aa, Caenorhabditis elegans
MDKTRALIDS

SEQ ID NO: 14 - protein, 19 aa, Caenorhabditis elegans
LEHLLEKDIITLEMRELIQ

SEQ ID NO: 15 - protein, 10 aa, Caenorhabditis elegans
  misc_feature (10)..(10): X is any amino acid.
GHLEDMLLTX

SEQ ID NO: 16 - protein, 19 aa, Caenorhabditis elegans
WDVLLSRELFRPHMIEDIQ

SEQ ID NO: 17 - protein, 10 aa, Caenorhabditis elegans
RDQARQLIID

SEQ ID NO: 18 - protein, 19 aa, Caenorhabditis elegans
AEVLVEGLVLQYLYQEGIL

SEQ ID NO: 19 - protein, 10 aa, Caenorhabditis elegans
LRKTMLLLDI

SEQ ID NO: 20 - protein, 41 aa, Caenorhabditis elegans
GSADNKQITEMLPGILNQLGPESLTHLKKLANNVTKLGPDG

SEQ ID NO: 21 - protein, 39 aa, Caenorhabditis elegans
TQIPLQARELIDTILVKGNAAANIFKNCLKEIDSTLYKN

SEQ ID NO: 22 - protein, 39 aa, Caenorhabditis elegans
TQTSLQARELIDTILVKGNIAATVFRNSLQEAEAVLYEH

SEQ ID NO: 23 - protein, 42 aa, Caenorhabditis elegans
TTGLRKTMLLLDILPSRGPKAFDTFLDSLQEFPWVREKLKKA

SEQ ID NO: 24 - protein, 40 aa, Caenorhabditis elegans
VGSFSQNVELLNLLPKRGPQAFDAFCEALRETKQGHLEDM

SEQ ID NO: 25 - protein, 40 aa, Caenorhabditis elegans
GTVREKRREIVKAVQRRGDVAFDAFYDALRSTGHEGLAEV

SEQ ID NO: 26 - protein, 42 aa, Caenorhabditis elegans
GSGSRRDQARQLIIDLETRGSQALPLFISCLEDTGQDMLASF

SEQ ID NO: 27 - protein, 40 aa, Caenorhabditis elegans
  misc_feature (34)..(34): X is any amino acid.
ATSRSRAGLLVDMCQDHPRGFQCLKESCKNEVGXEHLVDL

SEQ ID NO: 28 - protein, 40 aa, Caenorhabditis elegans
NATVMDKTRALIDSVIPKGAQACQICITYICEEDSYLAGT

SEQ ID NO: 29 - protein, 40 aa, Caenorhabditis elegans
DAKTEDKVRVMADSMQEKQRMAGQMLLQTFFNIDQLSPNK

SEQ ID NO: 30 - protein, 39 aa, Caenorhabditis elegans
PDAERRVRRLLLLVQSKGEAACQELLRCAQQTVSMPDPA

SEQ ID NO: 31 - protein, 38 aa, Caenorhabditis elegans
TRLERIANFLRIYRRQASELGPLIDFFNYNNQSHLADF

SEQ ID NO: 32 - protein, 152 aa, Homo sapiens
  misc_feature (132)..(143): Xaa is any amino acid
EKLAKLQAQVRIGGKGTARRKKKVVHRTATADDKKLQFSLKKLGVNNISGIEEVNMFTNQGTVIHFNNPKVQASLAANTFTITGHAETKQLTEMLPSILNQLGADSLTSLRRLAEALPKQSVDGKAPLATGXXXXXXXXXXXXNFDEASKNE

SEQ ID NO: 33 - protein, 147 aa, Caenorhabditis elegans
ERIKKLQAQQEHVRIGGKGTPRRKKKVIHKTAAADDKKLQSNLKKLSVTNIPGIEEVNMIKDDGTVIHFNNPKVQTSVPANTFSVTGSADNKQITEMLPGILNQLGPESLTHLKKLANNVKLGPDGKGEDEDVPELVGDFDAASKNE

SEQ ID NO: 34 - protein, 94 aa, Artificial Sequence
  Consensus sequence -- consensus between C. elegans and Human  BTF
EKLQAQVRIGGKGTRRKKKVHTAADDKKLQLKKLVNIGIEEVNMGTVIHFNNPKVQSANTFTGAKQTEMLPILNQLGSLTLLAKDGKFDASKNE

SEQ ID NO: 35 - RNA, 14 nt, Artificial Sequence
  Synthetic hairpin ribozyme.
nnnbngucnnnnnn